(19) United States
(12) Patent Application Publication
(10) Pub. No.: US 2003/0027171 A1
(43) Pub. Date: Feb. 6, 2003

(54) Cell cycle regulator protein

(76) Inventors: Chu-Wen Yang, Taipei (TW); Ann-Ping Tsou, Taipei (TW); Chin-Wen Chi, Taipei (TW); Ming-Ji Fann, Taipei (TW); Chen-Kung Chou, Taipei (TW)

Correspondence Address: Y. ROCKY TSAO, Fish & Richardson P.C., 225 Franklin Street, Boston, MA, 02110-2804, US

(21) Appl. No.: 10/051,409

(22) Filed: Jan. 18, 2002

Related U.S. Application Data
(60) Provisional application No. 60/262,885, filed on Jan. 19, 2001.

(51) Int. Cl. C12Q 1/68; C07H 21/04; C12N 9/22; C12P 21/02; C12N 5/06

(52) U.S. Cl. 435/6; 435/199; 536/23.2; 435/325; 435/320.1; 435/69.1

(57) ABSTRACT

This invention relates to new cell cycle regulatory proteins and nucleic acids encoding them.

### RELATED APPLICATION

This application claims the benefit of U.S. Provisional Patent Application No. 60/262,885, filed Jan. 19, 2001, the content of which is hereby incorporated by reference.

### BACKGROUND

A variety of genes that are over-expressed in tumor cells relative to healthy cells have been identified. It is expected that the identification of such genes will provide drug targets for anti-cancer drug development and for cancer diagnostics.

### SUMMARY

This invention is based on the discovery of a human cell cycle regulator protein that is over-expressed in hepatoma cells relative to normal adjacent tissues in a liver cancer patient. This protein is designated hepatoma up-regulated protein or HURP. The full-length human HURP cDNA (GenBank Accession No. AB076695) is shown below:

Human HURP eDNA Secquence

| | | | |
|---|---|---|---|
| | AGCAAACCAATCGCAAGCCTCGTTGAGTGGAAGGGGT | −181 | (SEQ |
| | GGGATCTTCCCCGGAAGTTTTGGTTAAAGCCCCTCCAATCAGCGGCTCGGTGCGGCAAGTTTGAATTTCGTGGAGGCTCGGGTTGTGAGG | −91 | ID |
| | GTTCCTGCTTCGGAGTCGGCGGTGGTCGTCCAGACCGAGTGTTCTTTACTTTTTGTTTGGTTGAGGTTTCACGCTAGAAGGTGGCTCAGG | −1 | NO: |
| | ATGTCTTCATCACATTTTGCCAGTCGACACAGGAAGGATATAAGTACTGAAATGATTAGAACTAAAATTGCTCATAGGAAATCACTGTCT | 90 | 6) |
| 1 | M S S S H F A S R H R K D I S T E M I R T K I A H R K S L S | | (SEQ |
| | CAGAAAGAAAATAGACATAAGGAATACGAACGAAATAGACACTTTGGTTTGAAAGATGTAAACATTCCAACCTTGGAAGGTAGAATTCTT | 180 | ID |
| 31 | Q K E N R H K E Y E R N R H F G L K D V N I P T L E G R I L | | NO: |
| | GTTGAATTAGATGAGACATCTCAAGAGCTTGTTCCAGAAAAGACCAATGTTAAGCCAAGGGCAATGAAAACTATTCTAGGTGATCAACGA | 270 | 4) |
| 61 | V E L D E T S Q E L V P E K T N V K P R A M K T I L G D Q R | | |
| | aAACAGATGCTCCAAAAATACAAAGAAGAAAAGCAACTTCAAAAATTGAAAGAGCAGAGAGAGAAAGCTAAACGAGGAATATTTAAAGTG | 360 | |
| 91 | K Q M L Q E Y K E E K Q L Q K L K E Q R E K A K R G I F K V | | |
| | GGTCGTTATAGACCTGATATGCCTTGTTTTCTTTTATCAAACCAGAATGCTGTGAAAGCTGAGCCAAAAAAGGCTATTCCATCTTCTGTA | 450 | |
| 121 | G R Y R P D K M P C F L L S N Q N A V K A E P K K A I P S S V | | |
| | CGGATTACAAGGTCAAAGGCCAAAGACCAAATGGAGCAGACTAAGATTGATAACGAGAGTGATGTTCGAGCAATCCGACCTGGTCCAAGA | 540 | |
| 151 | R I T R S K A K D Q M E Q T K I D N E S D V R A I R P G P R | | |
| | CAAACTTCTGAAAAGAAAGTGTCAGACAAAGAGAAAAAAGTTGTGCAGCCTGTAATGCCCACGTCGTTGAGAATGACTCGATCAGCTACT | 630 | |
| 181 | Q T S E K K V S D K E K K V V Q P V M P T S L R M T R S A T | | |
| | CAAGCAGCAAAGCAGGTTCCCAGAACAGTCTCATCTACCACAGCAAGAAAGCCAGTCACAAGAGCTGCTAATGAAAACGAACCAGAAGGA | 720 | |
| 211 | Q A A K Q V P R T V S S T T A R K P V T R A A N E N E P E G | | |
| | AAGGTGCCAAGTAAAGGAAGACCTGCCAAAAATGTAGAAACAAAACCCGACAAGGGTATTTCTTGTAAAGTCGATAGTGAAGAAAATACT | 810 | |
| 241 | K V P S K G R P A K N V E T K P D K G I S C K V D S E E N T | | |
| | TTGAATTCACAAACTAATGCAACAAGTGGAATGAATCCAGATGGAGTCTTATCAAAAATGGAAAACTTACCTGAGATAAATACTGCAAAA | 900 | |
| 271 | L N S Q T N A T S G M N P D G V L S K M E N L P E I N T A K | | |
| | ATAAAAGGGAAGAATTCCTTCGCACCTAAGGATTTTATGTTTCAGCCACTGGATGGTCTGAAGACCTATCAAGTAACACCTATGACTCCC | 990 | |
| 301 | I K G K N S F A P K D F M F Q P L D G L K T Y Q V T P M T P | | |
| | AGAAGTGCCAATGCTTTTTTGACACCCAGTTACACCTGGACTCCTTTAAAAACAGAAGTTGATGAGTCTCAAGCAACAAAAGAAATTTTG | 1080 | |
| 331 | R S A N A F L T P S Y T W T P L K T E V D E S Q A T K E I L | | |
| | GCACAAAAATGTAAAACTTACTCTACCAAGACAATACAGCAAGATTCAAATAAATTGCCATGTCCTTTGGGTCCTCTAACTGTTTGGCAT | 1170 | |
| 361 | A Q K C K T Y S T K T I Q Q D S N K L P C P L G P L T V W H | | |
| | GAAGAACATGTTTTAAATAAAAATGAAGCTACTACTAAAAATTTAAATGGCCTTCCAATAAAAGAAGTCCCATCACTTGAAAGAAATGAA | 1260 | |
| 391 | E E H V L N K N E A T T K N L N G L P I K E V P S L E R N E | | |
| | GGTCGAATTGCTCAGCCCCACCATGGTGTGCCATATTTCAGAAATATCCTCCAGTCAGAAACTGAGAAATTAACTTCACATTGCTTCGAG | 1350 | |
| 421 | G R I A Q P H H G V P Y F R N I L Q S E T E K L T S H C F E | | |
| | TGGGACAGGAAACTTGAATTGGACATTCCAGATGATGCTAAAGATCTTATTCGCACAGCAGTTGGTCAAACAAGACTCCTTATGAAGGAA | 1440 | |
| 451 | W D R K L E L D I P D D A K D L I R T A V G Q T R L L M K E | | |
| | AGGTTTAAACAGTTTGAAGGACTGGTTGATGATTGTGAATATAAACGAGGTATAAAGGAGACTACCTGTACAGATCTGGATGGATTTTGG | 1530 | |
| 481 | R F K Q F E G L V D D C E Y K R G I K E T T C T D L D G F W | | |
| | GATATGGTTAGTTTTCAGATAGAAGATGTAATCCACAAATTCAACAATCTGATCAAACTTGAGGAATCTGGGTGGCAAGTCAATAATAAT | 1620 | |
| 511 | D M V S F Q I E D V I H K F N N L I K L E E S G W Q V N N N | | |
| | ATGAATCATAATATGAACAAAAATGTCTTTAGGAAAAAAGTTGTCTCAGGTATAGCAAGTAAACCAAAACAGGATGATGCTGGAAGAATT | 1710 | |
| 541 | M N H N M N K N V F R K K V V S G I A S K P K Q D D A G R I | | |
| | GCAGCGAGAAATCGCCTAGCTGCCATAAAAAATGCAATGAGAGAGAGAATTAGGCAGGAAGAATGTGCTGAAACAGCAGTTTCTGTGATA | 1800 | |
| 571 | A A R N R L A A I K N A M R E R I R Q E E C A E T A V S V I | | |
| | CCAAAGGAAGTTGATAAAATAGTGTTCGATGCTGGATTTTTCAGAGTTGAAAGTCCTGTTAAATTATTCTCAGGACTTTCTGTCTCTTCT | 1890 | |
| 601 | P K E V D K I V F D A G F F R V E S P V K L F S G L S V S S | | |
| | GAAGGCCCTTCTCAAAGACTTGGAACACCTAAGTCTGTCAACAAAGCTGTATCTCAGAGTAGAAATGAGATGGGCATTCCACAACAAACT | 1980 | |
| 631 | E G P S Q R L G T P K S V N K A V S Q S R N E M G I P Q Q T | | |
| | ACATCACCAGAAAATGCCGGTCCTCAGAATACGAAAAGTGAACATGTGAAGAAGACTTTGTTTTTGAGTATTCCTGAAAGCAGGAGCAGC | 2070 | |
| 661 | T S P E N A G P Q N T K S E H V K K T L F L S I P E S R S S | | |
| | ATAGAAGATGCTCAGTGTCCTGGATTACCAGATTTAATTGAAGAAAACCATGTTGTAAATAAGACAGACTTGAAGGTGGATTGTTTATCC | 2160 | |
| 691 | I E D A Q C P G L P D L I E E N H V V N K T D L K V D C L S | | |
| | AGTGAGAGAATGAGTTTGCCTCTTCTTGCTGGTGGAGTAGCAGATGATATTAATACTAACAAAAAAGAAGGAATTTCAGATGTTGTGGAA | 2250 | |
| 721 | S E R M S L P L L A G G V A D D I N T N K K E G I S D V V E | | |
| | GGAATGGAACTGAATTCTTCAATTACATCACAGGATGTTTTGATGAGTAGCCCTGAAAAAATACAGCTTCACAAAATAGCATCTTAGAA | 2340 | |
| 751 | G M E L N S S I T S Q D V L M S S P E K N T A S Q N S I L E | | |
| | GAAGGGGAAACTAAAATTTCTCAGTCAGAACTATTTGATAATAAAAGTCTCACTACTGAATGCCACCTTCTTGATTCACCAGGTCTAAAC | 2430 | |
| 781 | E G E T K I S Q S E L F D N K S L T T E C H L L D S P G L N | | |
| | TGCAGTAATCCATTTACTCAGCTGGAGAGGAGACATCAAGAACATGCCAGACACATTTCTTTTGGTGGTAACCTGATTACTTTTTCACCT | 2520 | |
| 811 | C S N P F T Q L E R R H Q E H A R H I S F G G N L I T F S P | | |
| | CTACAACCAGGAGAATTTTGA 2541 | | |
| 841 | L Q P G E F * | | |
| | ATTTAAAAATAAATCCAAACATTTTCCTTCATATTATCAATGCTTATATATTCCTTAGACTATTGAAATTTTGGAGAAAATGTATTTGTG | 2631 | |
| | TTCACTTCTATAGCATATAATGTTTTAATATTCTGTGTTCATCAAAGTGTATTTTAGATATACTCTTTCTCAAGGGAAGTGGGGATATTT | 2721 | |
| | TGTACATTTTCAACACAGAATAAAAAATGTACTGTGCCTTG 2762 | | |

A mouse HURP gene has also been cloned. The full-length mouse HURP cDNA (GenBank Accession No. AB076696) is as follows:

Mouse HURP eDNA Sequence

| | | | |
|---|---|---|---|
| | AGTTTATAGTGTGTCGCTGCGTCGCCTAGC | −271 | (SEQ |
| | GGGTTTACCGCCTCCCTCCTCCCCCTCGCCCTCCCGCTCCCAACCCTTTGCCTTCCAAACAATTTAAATGTCGCACAGAACCAACCTATC | −181 | ID |
| | GCAAGCCTCGTTCGAGGGGAAGGGGCGGGAGCTTCCGGAAGTGTTGGCAAAGTCCCTCCAATCAGCGGCTGGCAGCGGGAAATTTCAGT | −9 | NO: |
| | TCCGTGAAGGGTCGGTCCGGGAGTTCCTTCTGGGGATCGGTGGAGTTTTCTGTGTTGGGAAATTGTTGTGGATCCAGAAACTGCTTCAGG | −1 | 5) |
| | ATGCTGGTGTCACGTTTTGCCAGTCGGTTTCGGAAAGACTCGAGCACTGAGATGGTTAGAACCAACTTGGCTCATAGAAAGTCTCTGTCT | 90 | |
| 1 | M L V S R F A S R F R K D S S T E M V R T N L A H R K S L S | | (SEQ |
| | CAGAAGGAGAACAGACACAGGGTGTATGAGCGAAACAGACACTTCGGTTTGAAGGACGTCAACATTCCACTGGAAGGGCGAGAGCTTGGT | 180 | |
| 31 | Q K E N R H R V Y E R N R H F G L K D V N I P L E G R E L G | | ID |
| | AATATACACGAGACATCGCAAGACCTCTCTCCAGAGAAGGCCAGCTCCAAAACAAGGTCAGTAAAAATGGTCCTGAGTGACCAACGGAAG | 270 | |
| 61 | N I H E T S Q D L S P E K A S S K T R S V K M V L S D Q R K | | NO: |
| | CAGCTCCTCCAGAAGTATAAGGAAGAAAAACAACTTCAAAAACTGAAAGAACAGCGAGAGAAAGCCAAACGTGGAGTGTTCAAAGTGGGT | 360 | |
| 91 | Q L L Q K Y K E E K Q L Q K L K E Q R E K A K R G V F K V G | | 2) |
| | CTCTATAGACCCGCTGCGCCTGGCTTTCTTGTCACAGACCAGAGGGGTGCGAAAGCTGAGCCAGAAAAGGCTTTTCCACATACTGGACGG | 450 | |
| 121 | L Y R P A A P G F L V T D Q R G A K A E P E K A F P H T G R | | |
| | ATTACAAGATCAAAGACCAAAGAATATATGGAGCAGACTAAGATTGGTAGCAGGAATGTTCCTAAAGCAACCCAGAGTGACCAAAGACAA | 540 | |
| 151 | I T R S K T K E Y M E Q T K I G S R N V P K A T Q S D Q R Q | | |
| | ACTTCTGAAAAACAACCATTAGACAGAGAGAGAAAAGTTATGCAGCCTGTGCTGTTCACGTCAGGGAAAGGGACTGAATCAGCGGCTACT | 630 | |
| 181 | T S E K Q P L D R E R K V M Q P V L F T S G K G T E S A A T | | |
| | CAGAGAGCCAAGCTGATGGCCCGAACAGTGTCATCCACTACAAGAAAGCCAGTCACAAGAGCCACGAATGAGAAAGGATCAGAAAGAATG | 720 | |
| 211 | Q R A K L M A R T V S S T T R K P V T R A T N E K G S E R M | | |
| | AGACCAAGTGGAGGGAGACCTGCCAAAAAACCAGAAGGCAAGCCGGACAAGGTCATTCCTTCCAAAGTTGAGCGGGACGAAAAGCATTTG | 810 | |
| 241 | R P S G G R P A K K P E G K P D K V I P S K V E R D E K H L | | |
| | GATTCGCAGACCAGGGAAACAAGTGAAATGGGTCTGCTCGGAGTCTTCCGAGAAGTGGAAAGCTTGCCTGCAACAGCCCCTGCCCAAGGG | 900 | |
| 271 | D S Q T R E T S E M G L L G V F R E V E S L P A T A P A Q G | | |
| | AAGGAAAGGAAGTCCTTTGCCCCCAAGCACTGTGTCTTCCAGCCCCCGTGTGGTCTGAAGAGCTACCAGGTGGCTCCCCTGAGCCCTAGA | 990 | |
| 301 | K E R K S F A P K H C V F Q P P C G L K S Y Q V A P L S P R | | |
| | AGTGCCAACGCTTTCCTGACACCCAATTGTGATTGGAACCAGTTAAGACCAGAAGTTTTTAGCACTACAACTCAAGACAAAGCAAATGAA | 1080 | |
| 331 | S A N A F L T P N C D W N Q L R P E V F S T T T Q D K A N E | | |
| | ATCTTGGTACAGCAAGGATTGGAGTCGCTAACAGACCGTAGTAAAGAACATGTCTTAAATCAGAAGGGCGCTTCTACTTCAGATTCAAAT | 1170 | |
| 361 | I L V Q Q G L E S L T D R S K E H V L N Q K G A S T S D S N | | |
| | CACGCTTCTGTGAAAGGAGTCCCATGCTCTGAAGGGAGCGAAGGCCAGACCTCTCAGCCCCCCACGATGTGCCATACTTCAGAAAAATC | 1260 | |
| 391 | H A S V K G V P C S E G S E G Q T S Q P P H D V P Y F R K I | | |
| | CTCCAATCAGAAACTGACAGGCTGACCTCGCACTGCCTGGAGTGGGAGGGGAAGCTGGACCTGGACATCTCTGATGAAGCTAAAGGTCTT | 1350 | |
| 421 | L Q S E T D R L T S H C L E W E G K L D L D I S D E A K G L | | |
| | ATCCGTACAACGGTTGGTCAAACAAGACTCCTTATCAAGGAGAGATTCAGACAGTTTGAAGGACTGGTGGACAACTGCGAGTATAAACGG | 1440 | |
| 451 | I R T T V G Q T R L L I K E R F R Q F E G L V D N C E Y K R | | |
| | GGTGAAAAGGAGACGACCTGCACAGATCTGGATGGATTCTGGGATATGGTTAGTTTTCAGGTCGATGATGTGAACCAGAAATTCAACAAC | 1530 | |
| 481 | G E K E T T C T D L D G F W D M V S F Q V D D V N Q K F N N | | |
| | CTGATCAAACTTGAGGCGTCAGGATGGAAAGACAGCAATAATCCAAGCAAAAAAGTCCTCCGGAAAAAATTGTGCCTGGTAGAACAAGC | 1620 | |
| 511 | L I K L E A S G W K D S N N P S K K V L R K K I V P G R T S | | |
| | AAAGCAAAGCAGGATGACGACGGACGAGCGGCAGCTAGGAGTCGCCTTGCTGCCATAAAGAATGCAATGAAAGGCAGGCCACAGCAGGAA | 1710 | |
| 541 | K A K Q D D D G R A A A R S R L A A I K N A M K G R P Q Q E | | |
| | GTGCAGGCCCACGCAGCAGCTCCGGAGACCACAAAGGAAGTTGACAAAATAGTGTTTGACGCTGGGTTTTTCAGAATCGAGAGCCCAGTG | 1800 | |
| 571 | V Q A H A A A P E T T K E V D K I V F D A G F F R I E S P V | | |
| | AAGTCATTCTCAGTCCTGTCTTCTGAACGTCGTTCTCAAAGATTTGGAACACCTCTGTCTGCCAGCAAAGTTGTGCCTGAGGGCAGGGCT | 1890 | |
| 601 | K S F S V L S S E R R S Q R F G T P L S A S K V V P E G R A | | |
| | GCAGGGGACCTTCTGAGACAGAAGATGCCACTGAAGAAGCCGGACCCTCAGAGCAGCAAGAGTGAGCATGTTGATCGGACGTTTTCAGAT | 1980 | |
| 631 | A G D L L R Q K M P L K K P D P Q S S K S E H V D R T F S D | | |
| | GGTCTTGAAAGCAGGTGCCACGTAGAAGACACCCCCTGTCCTGGAGAGCAAGATTCAAGTGACATAGAGCATGATGTAAATAAAATAAAT | 2070 | |
| 661 | G L E S R C H V E D T P C P G E Q D S S D I E H D V N K I N | | |
| | GTCAAGATGGATTGTTTCTCTGTTGAAACGAATTTGCCTCTTCCTGCTGGTGATGCTAATACCAATCAAAAAGAAGCAATCTCAGCCGTG | 2160 | |
| 721 | E G A S T A V T S Q D L L M S N P E T N T S S Q S N T S Q E | | |
| | GAAGCTGAGGCGTCGCAGTCAGTACTGTTACATAAAAGTCTCACTTCTGAATGCCACCTTCTTGAACCACCAGGCCTCAGCTGCACCAGC | 2340 | |
| 751 | E A E A S Q S V L L H K S L T S E C H L L E P P G L S C T S | | |
| | CCCTGCACTCGGGAGGAGACCAGACAGCCAGATCGCAGCAGACAGTTCTCCTTTGGAGGTGACCTCATTCTCTTCTCACCACTATGA | 2427 | |
| 781 | P C T R E E T R Q P D R S R Q F S F G G D L I L F S P L * | | |
| | CCCTGAAGGGAACACCAGGAGGGCTTTAAATTTAACATGACTTTTAATATTAATTTAAATAAACATTCAGTGCTCGCCTTTAATCCCAGC | 2517 | |
| | ACTCCGGGAGGTAGAGGCAGGCGGATTTCTGAGTTCGAGGCCAGCCTGGTCTACAGAGTGAGTTCCAGGACAGCCAGGACTATACAGAGA | 2607 | |
| | AACCCTGTCTCGAAAAACCAAAATAAATAAATAAATAAATAAACAAACAAACAAACAAA 2666 | | |

The nucleotide sequence encoding the human HURP protein (i.e., from the ATG start codon to the codon immediately before the stop codon in SEQ ID NO: 6) is designated SEQ ID NO: 3, and the nucleotide sequence encoding the mouse HURP protein (i.e., from the ATG start codon to the codon immediately before the stop codon in SEQ ID NO: 5) is designated SEQ ID NO: 1.

The invention features a pure polypeptide including an amino acid sequence at least 65% (e.g., at least 70, 75, 80, 85, 90, 95, 98, or 100%) identical to SEQ ID NO: 2 or 4. Once expressed in a cell, the polypeptide accelerates G2/M progression and promotes cell survival.

A “pure polypeptide” is a polypeptide free from other biological macromolecules, e.g., it is at least 75% (e.g., at least 80, 85, 95, or 99%) pure by dry weight. Purity can be measured by any appropriate standard method, for example, by column chromatography, polyacrylamide gel electrophoresis, or HPLC analysis.

The “percent identity” of two amino acid sequences is determined using the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. Where gaps exist between two sequences, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are used. See http://www.ncbi.nlm.nih.gov.

The polypeptides of the invention, once expressed in a cell, accelerate G2/M progression, i.e., a cell over-expressing HURP has higher DNA synthesis, or needs less time to progress from M phase to G1 phase when released from mitotic arrest in a serum-free medium. Such acceleration of G2/M progression by over-expression of HURP is determined as described in Example 5 below.

Expression of the polypeptides of the invention also promotes cell survival, i.e., in a medium supplemented with 0.5%, 1%, or 2% serum, a cell over-expressing HURP grows at a steady rate until reaching a plateau, while a cell that does not over-express HURP stops proliferating shortly after an initial, slow growth. Such promotion of cell survival by over-expression of HURP is determined as described in Example 6 below.

The polypeptides of the invention can be used to generate antibodies (either monoclonal or polyclonal) that specifically bind to HURP protein. These antibodies in turn are useful for detecting the presence and distribution of HURP in tissues and in cellular compartments. For example, such antibodies can be used to determine whether a cell is non-dividing or in the G2/M phase by determining the HURP protein distribution in the cell. Alternatively, they can be used to diagnose cancerous tissues (e.g., liver cancer tissues) by determining whether HURP protein is over-expressed in the tissue.

The invention also features an isolated nucleic acid encoding a polypeptide of the invention, and the complement of the nucleic acid. An example of a nucleic acid within the invention is an isolated nucleic acid that hybridizes under stringent conditions (i.e., hybridization at 65° C., 0.5X SSC, followed by washing at 45° C., 0.1X SSC) to SEQ ID NO: 1 or 3, or the complement of SEQ ID NO: 1 or 3. Such a nucleic acid can have at least 15 (e.g., at least 30, 50, 100, 200, 500, or 1000) nucleotides in length. The nucleic acids of the invention can be used to diagnose cancer (e.g., liver cancer) by determining whether HURP mRNA is being over-expressed in a tissue or cell. The nucleic acids can be used as primers in PCR-based detection methods, or as labeled probes in nucleic acid blots (e.g., Northern blots).

An “isolated nucleic acid” is a nucleic acid the structure of which is not identical to that of any naturally occurring nucleic acid or to that of any fragment of a naturally occurring genomic nucleic acid spanning more than three separate genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of different (i) DNA molecules, (ii) transfected cells, or (iii) cell clones, e.g., as these occur in a DNA library such as a cDNA or genomic DNA library.

In addition, the invention features a method of (1) expressing in a cell a transcript, i.e., transcript I, that hybridizes under above-described high stringency conditions to SEQ ID NO: 1 or 3, or (2) expressing in a cell a transcript, i.e., transcript II, that is complementary to transcript I. Transcript I, when expressed in a cell, can serve as an anti-sense RNA that binds to endogenous HURP mRNA to prevent it from being translated into a functional protein. Therefore, this method can be used in gene therapy for treating cancer (e.g., a liver cancer). Transcript II may encode a HURP protein, and when expressed in a cell, is translated into a HURP protein. Thus, this method can be used for production of a polypeptide of the invention, or for treating a patient having a disease associated with insufficient HURP gene expression.

The invention further features a diagnostic method for determining whether a patient has a cell proliferation disorder by comparing the level of HURP gene expression in a test sample from the patient with the level of HURP gene expression in a control sample from a normal person. A higher HURP gene expression level in the test sample indicates that the patient has a cell proliferation disorder associated with over-expression of the HURP gene, e.g., a liver cancer. A lower HURP gene expression level in the test sample indicates that the patient has a cell proliferation disorder associated with insufficient expression of the HURP gene.

The invention also features a method of identifying a candidate compound useful for treating a cell proliferation disorder. For instance, a library of compounds can be screened by treating a cell that expresses HURP gene with each compound, and detecting and comparing HURP gene expression levels in the presence and absence of the test compound. A compound that represses HURP gene expression is a candidate for treating a cell proliferation disorder associated with over-expression of the HURP gene, e.g., a liver cancer. A compound that enhances HURP gene expression is a candidate for treating a cell proliferation disorder associated with insufficient expression of the HURP gene.

The details of one or more embodiments of the invention are set forth in the accompanying description below. Other features, objects, and advantages of the invention will be apparent from the description, and from the claims.

### DETAILED DESCRIPTION

The invention relates to new cell cycle regulator proteins (hepatoma up-regulated proteins or HURP) and nucleic acids encoding them. The human and mouse HURP cDNAs have been cloned as described in Examples 1-2 below.

HURP is over-expressed in hepatocellular carcinoma cells relative to normal liver cells. In normal human tissues, HURP mRNA is only expressed at high levels in a subset of proliferating normal tissues such as thymus, colon and testis, but not liver. The endogenous level of HURP mRNA is tightly regulated during a cell cycle as illustrated by its elevated expression (1) in G2/M phase of the synchronized HeLa cells and (2) in regenerating mouse liver after partial hepatectomy. In addition to differential expression, the cellular distribution of HURP protein differs depending on the phase of a cell cycle. Immunofluorescence studies reveal that HURP localizes in cytosol during interphase of a cell cycle, and moves to the spindle poles during mitosis. Further, over-expression of HURP in 293T cells results in faster G2/M progression and enhanced cell growth in low serum medium.

The above observations indicate that (1) HURP regulates G2/M transition during the cell cycle, (2) over-expression of HURP leads to cancer by allowing cells to progress through the cell cycle with reduced dependence upon extracellular growth factors when normal cells would be blocked from doing so, and (3) inhibition of HURP expression or activity reverts cancer cells to a more normal phenotype. Thus, HURP is a new oncogene and therefore a new cancer drug target.

Uses of HURP

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express a HURP protein (e.g., via a recombinant expression vector in a host cell in gene therapy applications), to detect a HURP mRNA (e.g., in a biological sample) or a genetic alteration in a HURP gene, and to modulate HURP activity. The HURP proteins can be used to treat disorders characterized by insufficient or excessive production of a HURP substrate or production of HURP inhibitors. In addition, the HURP proteins can be used to screen for naturally occurring HURP substrates, to screen for drugs or compounds which modulate HURP activity, as well as to treat disorders characterized by insufficient or excessive production of HURP protein or production of HURP protein forms that have decreased, aberrant, or unwanted activity compared to HURP wild type protein (e.g., in liver cancer). Moreover, the anti-HURP antibodies of the invention can be used to detect and isolate HURP proteins, regulate the bioavailability of HURP proteins, and modulate HURP activity.

A method of evaluating a compound for the ability to interact with, e.g., bind, a subject HURP polypeptide is provided. The method includes: contacting the compound with the subject HURP polypeptide; and evaluating ability of the compound to interact with, e.g., to bind or form a complex with, the subject HURP polypeptide. This method can be performed in vitro, e.g., in a cell free system, or in vivo, e.g., in a two-hybrid interaction trap assay. This method can be used to identify naturally occurring molecules that interact with subject HURP polypeptide. It can also be used to find natural or synthetic inhibitors of a subject HURP polypeptide.

(a) Screening Assays

The invention provides methods (also referred to herein as “screening assays”) for identifying modulators, i.e., candidate or test compounds or agents (e.g., proteins, peptides, peptidomimetics, peptoids, small molecules, or other drugs) which bind to HURP proteins, have a stimulatory or inhibitory effect on, for example, HURP expression or HURP activity, or have a stimulatory or inhibitory effect on, for example, the expression or activity of a HURP substrate. Compounds thus identified can be used to modulate the activity of target gene products (e.g., HURP genes) in a therapeutic protocol, to elaborate the biological function of the target gene product, or to identify compounds that disrupt normal target gene interactions.

In one embodiment, the invention provides assays for screening candidate or test compounds which are substrates of a HURP protein or polypeptide or a biologically active portion thereof. In another embodiment, the invention provides assays for screening candidate or test compounds which bind to or modulate the activity of a HURP protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which is resistant to enzymatic degradation but which nevertheless remains bioactive; see, e.g., Zuckermann, R. N. et al. (1994) J. Med. Chem. 37:2678-85), spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer, or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), on beads (Lam (1991) Nature 354:82-84), chips (Fodor (1993) Nature 364:555-556), bacteria (Ladner, U.S. Pat. No. 5,223,409), spores (Ladner U.S. Pat. No. '409), plasmids (Cull et al. (1992) Proc Natl Acad Sci USA 89:1865-1869), or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one embodiment, an assay is a cell-based assay in which a cell which expresses a HURP protein or biologically active portion thereof is contacted with a test compound, and the ability of the test compound to modulate HURP activity is determined. Determining the ability of the test compound to modulate HURP activity can be accomplished by monitoring, for example, cell cycle-regulated cellular localization. The cell, for example, can be of mammalian origin, e.g., human.

The ability of the test compound to modulate HURP binding to a compound, e.g., a HURP substrate, or to bind to HURP can also be evaluated. This can be accomplished, for example, by coupling the compound, e.g., the substrate, with a radioisotope or enzymatic label such that binding of the compound, e.g., the substrate, to HURP can be determined by detecting the labeled compound, e.g., substrate, in a complex. Alternatively, HURP could be coupled with a radioisotope or enzymatic label to monitor the ability of a test compound to modulate HURP binding to a HURP substrate in a complex. For example, compounds (e.g., HURP substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a HURP substrate) to interact with HURP with or without the labeling of any of the interactants can be evaluated. For example, a microphysiometer can be used to detect the interaction of a compound with HURP without the labeling of either the compound or the HURP (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and HURP.

In yet another embodiment, a cell-free assay is provided in which a HURP protein or biologically active portion thereof is contacted with a test compound and the ability of the test compound to bind to the HURP protein or biologically active portion thereof is evaluated. Preferred biologically active portions of the HURP proteins to be used in assays of the present invention include fragments which participate in interactions with non-HURP molecules, e.g., fragments with high surface probability scores.

Soluble and/or membrane-bound forms of isolated proteins (e.g., HURP proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy- 1-propane sulfonate (CHAPSO), or N-dodecyl-N,N-dimethyl-3-ammonio- 1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor.’ Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the HURP protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either HURP, an anti-HURP antibody or its target molecule to facilitate separation of complexed from uncomplexed forms of one or both of the proteins, as well as to accommodate automation of the assay. Binding of a test compound to a HURP protein, or interaction of a HURP protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/HURP fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or HURP protein, and the mixture incubated under conditions conducive to complex formation (e.g., at physiological conditions for salt and pH). Following incubation, the beads or microtiter plate wells are washed to remove any unbound components, the matrix immobilized in the case of beads. Complexes are determined either directly or indirectly, for example, as described above. Alternatively, the complexes can be dissociated from the matrix, and the level of HURP binding or activity determined using standard techniques.

Other techniques for immobilizing either a HURP protein or a target molecule on matrices include using conjugation of biotin and streptavidin. Biotinylated HURP protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with HURP protein or target molecules but which do not interfere with binding of the HURP protein to its target molecule. Such antibodies can be derivatized to the wells of the plate, and unbound target or HURP protein trapped in the wells by antibody conjugation. Methods for detecting such complexes, in addition to those described above for the GST-immobilized complexes, include immunodetection of complexes using antibodies reactive with the HURP protein or target molecule, as well as enzyme-linked assays which rely on detecting an enzymatic activity associated with the HURP protein or target molecule.

Alternatively, cell free assays can be conducted in a liquid phase. In such an assay, the reaction products are separated from unreacted components by any of a number of standard techniques including but not limited to differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: N.Y.); and immunoprecipitation (see, for example, Ausubel, F. et al., eds. (1999) Current Protocols in Molecular Biology, J. Wiley: N.Y.). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the HURP protein or biologically active portion thereof with a known compound which binds HURP to form an assay mixture, contacting the assay mixture with a test compound, and determining the ability of the test compound to interact with a HURP protein, where determining the ability of the test compound to interact with a HURP protein includes determining the ability of the test compound to preferentially bind to HURP or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the HURP genes herein identified. In an alternative embodiment, the invention provides methods for determining the ability of the test compound to modulate the activity of a HURP protein through modulation of the activity of a downstream effector of a HURP target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form a complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases where it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner is anchored onto a solid surface (e.g., a microtiter plate), while the non-anchored species is labeled either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface, e.g., using a labeled antibody specific for the initially non-immobilized species. The antibody, in turn, can be directly labeled or indirectly labeled with, e.g., a labeled anti-Ig antibody. Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected, e.g., using an immobilized antibody specific for one of the binding components to anchor any complexes formed in solution and a labeled antibody specific for the other partner to detect anchored complexes. Again, depending upon the order of addition of reactants to the liquid phase, test compounds that inhibit complex formation or that disrupt preformed complexes can be identified.

In an alternate embodiment, a homogeneous assay can be used. For example, a preformed complex of the target gene product and the interactive cellular or extracellular binding partner product is prepared so that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the HURP proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300) to identify other proteins, which bind to or interact with HURP (“HURP-binding proteins” or “HURP-bp”) and are involved in HURP activity. Such HURP-bps can be activators or inhibitors of signals by the HURP proteins or HURP targets as, for example, downstream elements of a HURP-mediated signaling pathway.

The two-hybrid system is based on the modular nature of most transcription factors, which consist of separable DNA-binding and activation domains. Briefly, the assay utilizes two different DNA constructs. In one construct, the gene that codes for a HURP protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively, the HURP protein can be fused to the activator domain.) If the “bait” and the “prey” proteins are able to interact, in vivo, forming a HURP-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein that interacts with the HURP protein.

In another embodiment, modulators of HURP expression are identified. For example, a cell or cell free mixture is contacted with a candidate compound and the expression of HURP mRNA or protein evaluated relative to the level of expression of HURP mRNA or protein in the absence of the candidate compound. When expression of HURP mRNA or protein is greater in the presence of the candidate compound than in its absence, the candidate compound is identified as a stimulator of HURP mRNA or protein expression. Alternatively, when expression of HURP mRNA or protein is less (statistically significantly less) in the presence of the candidate compound than in its absence, the candidate compound is identified as an inhibitor of HURP mRNA or protein expression. The level of HURP mRNA or protein expression can be determined by methods described herein for detecting HURP mRNA or protein.

In another aspect, the invention pertains to a combination of two or more of the assays described herein. For example, a modulating agent can be identified using a cell-based or a cell free assay, and the ability of the agent to modulate the activity of a HURP protein can be confirmed in vivo, e.g., in an animal such as an animal model for hepatocellular carcinoma.

This invention further pertains to novel agents identified by the above-described screening assays. Accordingly, it is within the scope of this invention to further use an agent identified as described herein (e.g., a HURP modulating agent, an antisense HURP nucleic acid molecule, a HURP-specific antibody, or a HURP-binding partner) in an appropriate animal model to determine the efficacy, toxicity, side effects, or mechanism of action, of treatment with such an agent. Furthermore, novel agents identified by the above-described screening assays can be used for treating cancers, e.g., liver cancer.

(b) Use of HURP Molecules as Surrogate Markers

The HURP molecules of the invention are also useful as markers of disorders or disease states, as markers for precursors of disease states, as markers for predisposition of disease states, as markers of drug activity, or as markers of the pharmacogenomic profile of a subject. Using the methods described herein, the presence, absence and/or quantity of the HURP molecules of the invention may be detected, and may be correlated with one or more biological states in vivo. For example, the HURP molecules of the invention may serve as surrogate markers for one or more disorders or disease states or for conditions leading up to disease states. As used herein, a “surrogate marker” is an objective biochemical marker which correlates with the absence or presence of a disease or disorder, or with the progression of a disease or disorder (e.g., with the presence or absence of a liver tumor). The presence or quantity of such markers is independent of the disease. Therefore, these markers may serve to indicate whether a particular course of treatment is effective in lessening a disease state or disorder. Surrogate markers are of particular use when the presence or extent of a disease state or disorder is difficult to assess through standard methodologies (e.g., early stage tumors), or when an assessment of disease progression is desired before a potentially dangerous clinical endpoint is reached (e.g., an assessment of cardiovascular disease may be made using cholesterol levels as a surrogate marker, and an analysis of HIV infection may be made using HIV RNA levels as a surrogate marker, well in advance of the undesirable clinical outcomes of myocardial infarction or fully-developed AIDS). Examples of the use of surrogate markers in the art include those described in Koomen et al. (2000) J. Mass. Spectrom. 35: 258-264; and James (1 994) AIDS Treatment News Archive 209.

The HURP molecules of the invention are also useful as pharmacodynamic markers. As used herein, a “pharmacodynamic marker” is an objective biochemical marker which correlates specifically with drug effects. The presence or quantity of a pharmacodynamic marker is not related to the disease state or disorder for which the drug is being administered; therefore, the presence or quantity of the marker is indicative of the presence or activity of the drug in a subject. For example, a pharmacodynamic marker may be indicative of the concentration of the drug in a biological tissue, in that the marker is either expressed or transcribed or not expressed or transcribed in that tissue in relationship to the level of the drug. In this fashion, the distribution or uptake of the drug may be monitored by the pharmacodynamic marker. Similarly, the presence or quantity of the pharmacodynamic marker may be related to the presence or quantity of the metabolic product of a drug, such that the presence or quantity of the marker is indicative of the relative breakdown rate of the drug in vivo. Pharmacodynamic markers are of particular use in increasing the sensitivity of detection of drug effects, particularly when the drug is administered in low doses. Since even a small amount of a drug may be sufficient to activate multiple rounds of marker (e.g., a HURP marker) transcription or expression, the amplified marker may be in a quantity which is more readily detectable than the drug itself. Also, the marker may be more easily detected due to the nature of the marker itself; for example, using the methods described herein, anti-HURP antibodies may be employed in an immune-based detection system for a HURP protein marker, or HURP-specific radiolabeled probes may be used to detect a HURP mRNA marker. Furthermore, the use of a pharmacodynamic marker may offer mechanism-based prediction of risk due to drug treatment beyond the range of possible direct observations. Examples of the use of pharmacodynamic markers in the art are described in Matsuda et al. U.S. Pat. No. 6,033,862; Hattis et al. (1991) Env. Health Perspect. 90: 229-238; Schentag (1999) Am. J. Health-Syst. Pharm. 56 Suppl. 3: S21-S24; and Nicolau (1999) Am, J Health-Syst. Pharm. 56 Suppl. 3: S16-S20.

The HURP molecules of the invention are also useful as pharmacogenomic markers. As used herein, a “pharmacogenomic marker” is an objective biochemical marker which correlates with a specific clinical drug response or susceptibility in a subject (see, e.g., McLeod et al. (1999) Eur. J. Cancer 35:1650-1652). The presence or quantity of the pharmacogenomic marker is related to the predicted response of the subject to a specific drug or class of drugs prior to administration of the drug. By assessing the presence or quantity of one or more pharmacogenomic markers in a subject, a drug therapy which is most appropriate for the subject, or which is predicted to have a greater degree of success, may be selected. For example, based on the presence or quantity of RNA, or protein (e.g., HURP protein or RNA) for specific tumor markers in a subject, a drug or course of treatment may be selected which is optimized for the treatment of the specific tumor likely to be present in the subject. Similarly, the presence or absence of a specific sequence mutation in HURP DNA may correlate with HURP drug response. The use of pharmacogenomic markers therefore permits the application of the most appropriate treatment for each subject without having to administer the therapy.

(c) Pharmacogenomics

The HURP molecules of the present invention, as well as agents, or modulators which have a stimulatory or inhibitory effect on HURP activity (e.g., HURP gene expression) as identified by a screening assay described herein can be administered to individuals to treat (prophylactically or therapeutically) HURP associated disorders (e.g., liver cancer) associated with aberrant or unwanted HURP activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a HURP molecule or HURP modulator as well as tailoring the dosage and/or therapeutic regimen of treatment with a HURP molecule or HURP modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23:983-985 and Linder, M. W. et al. (1997) Clin. Chem. 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association,” relies primarily on a high-resolution map of the human genome consisting of already known gene-related markers (e.g., a “bi-allelic” gene marker map which consists of 60,000- 100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drug's target is known (e.g., a HURP protein of the present invention), all common variants of that gene can be fairly easily identified in the population, and it can be determined if having one version of the gene versus another is associated with a particular drug response.

Alternatively, a method termed the “gene expression profiling”, can be utilized to identify genes that predict drug response. For example, the gene expression of an animal dosed with a drug (e.g., a HURP molecule or HURP modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of an individual. This knowledge, when applied to dosing or drug selection, can avoid adverse reactions or therapeutic failure and thus enhance therapeutic or prophylactic efficiency when treating a subject with a HURP molecule or HURP modulator, such as a modulator identified by one of the exemplary screening assays described herein.

The present invention further provides methods for identifying new agents, or combinations, that are based on identifying agents that modulate the activity of one or more of the gene products encoded by one or more of the HURP genes of the present invention, where these products may be associated with resistance of the cells to a therapeutic agent. Specifically, the activity of the proteins encoded by the HURP genes of the present invention can be used as a basis for identifying agents for overcoming agent resistance. By blocking the activity of one or more of the resistance proteins, target cells, e.g., human cells, will become sensitive to treatment with an agent that the unmodified target cells were resistant to.

Monitoring the influence of agents (e.g., drugs) on the expression or activity of a HURP protein can be applied in clinical trials. For example, the effectiveness of an agent determined by a screening assay as described herein to increase HURP gene expression, protein levels, an HURP activity can be monitored in clinical trials of subjects exhibiting decreased HURP gene expression, protein levels, or HURP activity. Alternatively, the effectiveness of an agent determined by a screening assay to decrease HURP gene expression, protein levels, or an HURP activity can be monitored in clinical trials of subjects exhibiting increased HURP gene expression, protein levels, or HURP activity. In such clinical trials, the expression or activity of a HURP gene, and preferably other genes that have been implicated in, for example, a HURP-associated disorder can be used as a “read out” or marker of the phenotype of a particular cell.

Without further elaboration, it is believed that one skilled in the art can, based on the above disclosure and the example below, utilize the present invention to its fullest extent. The following examples are to be construed as merely illustrative of how one skilled in the art can isolate and use the polypeptides and nucleic acids of the invention, and are not limitative of the remainder of the disclosure in any way. All publications cited in this disclosure are hereby incorporated by reference.

### EXAMPLES

Example 1 Identification of HURP gene

An NCBI UniGene database search for Expressed Sequence Tags (ESTs) expressed in human liver-related cDNA libraries resulted in a set of 137,142 sequences. In addition, seven cDNA libraries were constructed from one normal adult liver tissue and three pairs of HCC tumor tissues and their adjacent tissues. A total of 5,615 sequences were collected from these seven libraries (http://lestdb.nhri.org.tw). According to the tissue origin of the cDNA libraries, these 142,757 ESTs were further grouped into 5 categories: fetal liver/spleen (116,698); normal adult liver tissues (19,944); HCC-tumor (hepatoma) tissues (2,694); tumor-adjacent normal tissues (2,457) and HCC cell lines (964). Identities of these ESTs were searched against the UniGene Build#79 and Genbank (version of May 16, 1999) by using the NCBI BLAST software version 2.0. Every EST was assigned to a particular UniGene group when it shared 85% sequence identity within a stretch of 100 base pairs from a known gene. 256 UniGene groups that are only present in cDNA libraries of human HCC tissue were identified. Further, gene expression profiles of these 256 UniGene groups were searched for in the microarray database established by Iyer, et al. (Science, 1999, 283: 83-87), which contains expression profiles of 8,600 genes of normal human fibroblasts after serum stimulation. Gene expression profiles were found for 32 UniGene groups, 12 of which were highly regulated during serum-induced cell cycle progression. Subsequent examination of the expression of 8 ESTs in six pairs of HCC tissues using RT-PCR showed five genes, including N32765, had elevated expression in hepatoma tissues relative to its adjacent normal tissues (Table 1 below). Among these 5 genes, N32765 showed the most distinct pattern of expression. The protein encoded by N32765 was designated as HURP.

TABLE 1
Eight genes, identified from bioinfomatics searches, showed up-regulated
transcription in 6 pairs of HCC samples

| | Level of expression in HCC: |
|---|---|
| | ratio of T > N in 6 pairs of |
| Clone ID/Gene name | HCC* |
| N32765 (HURP) | 6/6 |
| N69904 | 5/6 |
| AA019977 | 5/6 |
| Carboxypeptidase D | 3/6 |
| Palmitoyl-protein thioesterase | 3/6 |
| Amine oxide, copper-containing | 3/6 |
| (AOC3) | |
| Presenilin 1 | 2/6 |
| Shc adaptor homolog | 1/6 |

*Expression level of 8 genes in 6 pairs of HCC samples was detected by RT-PCR. T > N means expression of the gene is higher in the tumor tissues (T) than in adjacent normal tissue (N).

Example 2 Cloning of human and mouse HURP genes

Cloning of full-length HURP cDNAs of human and mouse origins was accomplished by assembling EST clones using the Sequencher software, PCR cloning, cDNA library screening, and 5′ RACE. Human full-length cDNA of HURP was isolated from Hep3B, a cell line derived from HCC. Thirteen mouse EST clones (AI592008, C88298, AI510131, AA162837, AI1550612, AA511899, AI552952, C78700, AA212615, AI605993, AI482307, AI427201, and AI563636) showed homology with human HURP sequences. Assembly of these ESTs generated a DNA fragment of 1759 base pairs with an overall homology of 55% with human HURP. The missing 3′ fragment (approximately 600 base pairs) was subsequently cloned from a mouse cDNA library, and a longer 5′ end was cloned by 5′ RACE.

The human and mouse HURP cDNAs encode polypeptides of 846 and 808 amino acids, respectively. They share 72% similarity at the nucleotide sequence level, and 66% similarity at the amino acid sequence level. Human HURP is mapped to chromosome 14q22-23. Comparison of the cDNA sequence and the genomic sequence of chromosome 14 shows that HURP is organized into nineteen exons displaying non-canonical intron/exon and exon/lintron borders. Motif analysis reveals that HURP contains a nuclear localization signal (NLS), a putative leucine rich nuclear export signal (NES), two destruction boxes (D-box), a KEN box, and a coiled-coil domain.

Example 3 HURP Antibodies

Human HURP cDNA was subcloned into pET32 vector (Novagen) and expressed in E. coli as His-tagged fusion protein. The protein was expressed as inclusion body, and was solubilized with 2 M urea. The solubilized protein was partially purified by nickel agarose under denaturing conditions. The purified protein was then dialyzed to remove the denaturant as described in the manufacturer's manual. The recombinant protein was then injected to mice to raise polyclonal antibodies, which were used for Western blot analysis and immunofluorescence. Antibody to FLAG (M2 from Kodak) was used for detection of HURP protein in stable transfectants.

Example 4 Expression of HURP is cell cycle regulated

To confirm whether the expression of HURP is cell cycle regulated as shown in microarray database, expression of HURP in synchronized HeLa cells was examined. HeLa cells were synchronized in G₁/S by thymidine/aphidicolin block, and total RNA was extracted from cells harvested at various time points after release from G₁/S transition for quantitative RT-PCR analysis. Unexpectedly, FACS analysis shows that the cells entered the S phase at 6 h and reached the highest mitotic index at 12 h. The level of HURP transcripts was low at the G₁/S transition. It rose steadily as the cells progressed through S-phase and G₂/M, and peaked at the time the cells exited from mitosis.

Example 5 Over-expression of HURP accelerates G₂/M progression

To examine whether expression of HURP gene in G₂/M exerts any regulatory function, HURP gene was ectopically over-expressed in human 293T cells. Unexpectedly, higher DNA synthesis was detected in HURP-transiently transfected cells (68% cells in S phase) than in parental 293T cells (35% cells in S phase). 293T cells stably transfected with HURP were subsequently established. Several stable transfectants expressing increased levels of HURP protein were selected and characterized. The cell cycle progression of both the parental 293T and a HURP-transfectant were analyzed and compared at various points after release from nocodazole-induced mitotic arrest. Unexpectedly, when cells were cultured in serum free medium, the stable transfectant took less time (2 h) to progress from M phase to G1 phase than the parental cell line (4 h).

Example 6 Over-expression of HURP promotes cell survival in low serum medium

To find out whether over-expression of HURP confers any growth advantage of cells, growth of parental 293T cells and HURP-stable transfectants in low serum medium was examined. Both cell lines grew very poorly in serum-free medium, and failed to proliferate. The parental 293T cells grew slowly in medium containing 2% serum during the first 24 h, and stopped growing the next day. Unexpectedly, the HURP-stable transfectants proliferated at a steady rate in media containing 0.5%, 1%, or 2% serum, and reached plateau with different saturation densities after 4 days in the respective culture medium. These results strongly indicate that over-expressed HURP protein acts in the capacity of a weak oncogene in supporting cell growth.

A number of embodiments of the invention have been described. Nevertheless, it will be understood that various modifications may be made without departing from the spirit and scope of the invention. Accordingly, other embodiments are within the scope of the following claims.

What is claimed is:

1. A pure polypeptide comprising an amino acid sequence at least 65% identical to SEQ ID NO: 2 or 4, wherein the polypeptide, once over-expressed in a cell, accelerates G₂/M progression and promotes cell survival.

2. The polypeptide of claim 1, wherein the amino acid sequence is at least 70% identical to SEQ ID NO: 4.

3. The polypeptide of claim 2, wherein the amino acid sequence is at least 80% identical to SEQ ID NO: 4.

4. The polypeptide of claim 3, wherein the amino acid sequence is at least 90% identical to SEQ ID NO: 4.

5. The polypeptide of claim 4, wherein the amino acid sequence is SEQ ID NO: 4.

6. An isolated nucleic acid encoding the polypeptide of claim 1, or a 2 complementary sequence thereof.

7. An isolated nucleic acid encoding the polypeptide of claim 2, or a complementary sequence thereof.

8. An isolated nucleic acid encoding the polypeptide of claim 3, or a complementary sequence thereof.

9. An isolated nucleic acid encoding the polypeptide of claim 4, or a complementary sequence thereof.

10. An isolated nucleic acid encoding the polypeptide of claim 5, or a complementary sequence thereof.

11. An antibody which selectively binds to the polypeptide of claim 1.

12. An antibody which selectively binds to the polypeptide of claim 2.

13. An antibody which selectively binds to the polypeptide of claim 3.

14. An antibody which selectively binds to the polypeptide of claim 4.

15. An antibody which selectively binds to the polypeptide of claim 5.

16. An isolated nucleic acid that hybridizes under high stringency conditions to SEQ ID NO: 1 or 3, or a complementary sequence thereof.

17. A method of expressing a transcript in a cell, the method comprising:
introducing a vector into a cell, the vector containing a nucleic acid encoding a transcript; and
expressing the transcript in the cell;
wherein the transcript hybridizes under high stringency conditions to SEQ ID NO: 1 or 3, or a complementary sequence thereof.

18. The method of claim 17, wherein the transcript encodes the polypeptide of claim 1.

19. A method of determining whether a patient has a cell proliferation disorder, the method comprising:
providing a test sample from a patient suspected of having a cell proliferation disorder, and
detecting hepatoma-up-regulated protein gene expression in the test sample,
wherein a level of hepatoma-up-regulated protein gene expression in the test sample different from a level of hepatoma-up-regulated protein gene expression in a control sample from a normal person indicates that the patient has a cell proliferation disorder.

20. The method of claim 19, wherein the level of hepatoma-up-regulated protein gene expression in the test sample is higher than the level of hepatoma-up-regulated protein gene expression in the control sample.

21. The method of claim 20, wherein the cell proliferation disorder is a cancer.

22. The method of claim 21, wherein the cancer is hepatocellular carcinoma or cervical carcinoma.

23. The method of claim 19, wherein the level of hepatoma-up-regulated protein gene expression in the test sample is lower than the level of hepatoma-up-regulated protein gene expression in the control sample.

24. A method of identifying a candidate compound useful for treating a cell proliferation disorder, the method comprising detecting hepatoma-up-regulated protein gene expression in the presence of a test compound, wherein a level of hepatoma-up-regulated protein gene expression in the presence of the test compound different from a level of hepatoma-up-regulated protein gene expression in the absence of the test compound indicates that the test compound is a candidate useful for treating a cell proliferation disorder.

25. The method of claim 24, wherein the level of hepatoma-up-regulated protein gene expression in the presence of the test compound is lower than the level of hepatoma-up-regulated protein gene expression in the absence of the test compound.

26. The method of claim 25, wherein the cell proliferation disorder is a cancer.

27. The method of claim 26, wherein the cancer is hepatocellular carcinoma or cervical carcinoma.

28. The method of claim 24, wherein the level of hepatoma-up-regulated protein gene expression in the presence of the test compound is higher than the level of hepatoma-up-regulated protein gene expression in the absence of the test compound.
